(19) United States
(12) Patent Application Publication
(10) Pub. No.: US 2025/0255644 A1
(43) Pub. Date: Aug. 14, 2025

(54) TROCAR INCLUDING CANNULA

(71) Applicant: CILAG GMBH INTERNATIONAL, ZUG (CH)

(72) Inventors: Uri BARAK, Nesher (IL); Roy ZOHAR, Akko (IL); Shai NACHMIAS, Nahariya (IL); Hagay BOTANSKY, Haifa (IL)

(21) Appl. No.: 19/111,435

(22) PCT Filed: Sep. 13, 2023
(86) PCT No.: PCT/IL2023/050998

§ 371 Date: Mar. 13, 2025

Related U.S. Application Data
(60) Provisional application No. 63/409,231, filed on Sep. 23, 2022.
(60) Provisional application No. 63/409,229, filed on Sep. 23, 2022.

(51) Int. Cl. A61B 17/34

(52) U.S. Cl. CPC A61B 17/3421; A61B 17/3494; A61B 2017/347

(57) ABSTRACT

A trocar sleeve comprising: a cannula having a distal end and a proximal end, a housing having a distal end and a proximal end, the proximal end of the housing attached to the distal end of the cannula, and a lumen extending from the distal end of the cannula to the proximal end of the housing, wherein the cannula and housing are made of a biocompatible material designed to withstand a plurality of sterilization cycles.

### RELATED APPLICATIONS

This application claims the benefit of priority of U.S. Provisional Patent Application No. 63/409,231 filed on Sep. 23, 2022, and of U.S. Provisional Patent Application No. 63/409,229 filed on Sep. 23, 2022 the contents of which are incorporated herein by reference in their entirety.

### FIELD AND BACKGROUND OF THE INVENTION

The present invention, in some embodiments thereof, relates to medical devices for minimally invasive surgery and, more particularly, but not exclusively, to a trocar assembly.

Trocars are used during minimally invasive surgery, such as laparoscopic surgery. Trocars are placed through the skin of the body of a patient into a cavity, for example, abdomen or chest. The trocars provide access points into the cavity for tools such as graspers, scissors, staplers, and cameras.

### SUMMARY OF THE INVENTION

According to a first aspect, a trocar sleeve comprises: a cannula having a distal end and a proximal end, a housing having a distal end and a proximal end, the proximal end of the housing attached to the distal end of the cannula, and a lumen extending from the distal end of the cannula to the proximal end of the housing, wherein the cannula and housing are made of a biocompatible material designed to withstand a plurality of sterilization cycles.

In a further implementation form of the first aspect, the biocompatible materials is selected from a group comprising: polyetheretherketone (PEEK), and polyphenylsulfone (PPSU).

In a further implementation form of the first aspect, the biocompatible materials is designed to withstand at least two sterilization cycles.

In a further implementation form of the first aspect, the trocar sleeve has a smooth internal surface and external surface that excludes narrow features that trap debris and designed for flow of washing fluids to flow through for removal of debris for sterilization.

In a further implementation form of the first aspect, a range of an internal diameter of the lumen is 7.1-7.3 mm for an obturator or instrument with an external diameter of 5 mm, and 14.1-14.3 mm for an obturator or instrument with an external diameter of 12 mm.

In a further implementation form of the first aspect, an internal diameter of the lumen is selected to provide a clearance of about 0.3 mm from an outer diameter of an obturator placed within the lumen.

In a further implementation form of the first aspect, a range of an outer diameter of the cannula is 9.45-10.6 mm for an obturator or instrument with an external diameter of 5 mm, and 16.75-17.7 mm for an obturator or instrument with an external diameter of 12 mm.

In a further implementation form of the first aspect, an outer diameter of the housing ranges from 44.8-47.0 mm.

In a further implementation form of the first aspect, a working length of the trocar sleeve ranges from about 45 mm to 65 mm for pediatric use.

In a further implementation form of the first aspect, a working length of the trocar sleeve ranges from 60-150 mm.

In a further implementation form of the first aspect, further comprising at least one protruding feature designed for securing thread that is stitched to skin of a subject when the trocar sleeve is in use and positioned through the skin.

In a further implementation form of the first aspect, the cannula includes a plurality of raised features designed to engage a locking mechanism of a depth limiter that fixes location of the cannula relative to the depth limiter.

In a further implementation form of the first aspect, a distal end portion of the cannula excludes the raised features.

In a further implementation form of the first aspect, the plurality of raised features are spaced apart along a long axis of the cannula defining a resolution for fixing the location of the cannula relative to the depth limiter.

In a further implementation form of the first aspect, an external diameter of the cannula varies across a length of the cannula, the external diameter decreases from the proximal end of the cannula to the distal end of the cannula.

In a further implementation form of the first aspect, an external surface of the cannula includes a first guide element set for engaging a corresponding second guide element of a depth limiter, wherein the cannula is insertable into a lumen of the depth limiter by engaging the first guide element with the second guide element, and the trocar cannula is prevented from being inserted into the lumen of the depth limiter when the first guide element does not engage the second guide element.

In a further implementation form of the first aspect, the first guide element engaging the second guide element prevent rotation of the cannula within the depth limiter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic of a trocar assembly that includes a trocar sleeve designed for multiple uses, in accordance with some embodiments of the present invention;

FIG. 2 is a schematic of an exemplary trocar sleeve designed for multiple uses, in accordance with some embodiments of the present invention;

FIG. 3 is a schematic of an exemplary trocar sleeve designed for multiple uses and designed to engage with depth limiter, in accordance with some embodiments of the present invention;

FIG. 4 is a schematic of a bottom view (i.e., from distal to proximal) of trocar sleeve designed for multiple uses, in accordance with some embodiments of the present invention;

FIG. 5 is a schematic of a top view (i.e., from proximal to distal) of trocar sleeve designed for multiple uses, in accordance with some embodiments of the present invention;

FIG. 6 is a schematic of an exemplary depth limiter, in accordance with some embodiments of the present invention;

FIG. 7 is a schematic of an exemplary sealing element that engages with trocar sleeve, in accordance with some embodiments of the present invention;

FIG. 8 is a schematic of an exemplary obturator that engages with trocar sleeve, in accordance with some embodiments of the present invention; and

FIG. 9 is a schematic of an exploded view of trocar assembly, in accordance with some embodiments of the present invention.

### DESCRIPTION OF SPECIFIC EMBODIMENTS OF THE INVENTION

The present invention, in some embodiments thereof, relates to medical devices for minimally invasive surgery and, more particularly, but not exclusively, to a trocar assembly.

As used herein, the terms proximal and distal are used with reference to a user using the trocar assembly on a subject. Proximal refers to the portions of the trocar assembly that are closer to the user during use. Distal refers to the portions of the trocar assembly that are further away from the user during use.

An aspect of some embodiments of the present invention relates to a trocar sleeve for performing minimally invasive surgery, for example, laparoscopic surgery on an abdomen, chest, joint, and the like. The trocar sleeve include a cannula having a distal end and a proximal end, and a housing having a distal end and a proximal end. The proximal end of the housing is attached to the distal end of the cannula, for example, assembled, or formed as a single piece such as by injection molding. A lumen extends from the distal end of the cannula to the proximal end of the housing. The lumen is designed to engage with an obturator. The cannula and housing are made of a biocompatible material designed to withstand multiple sterilization cycles, for example, polyetheretherketone (PEEK), and polyphenylsulfone (PPSU). The biocompatible material is designed to withstand 2 or more sterilization cycles, for example, about 50, or 100, or 250, or 500, or 1000, or any other number of sterilization cycles. The biocompatible material is designed to withstand sterilization cycles performed, for example, by an autoclave device. The trocar sleeve has a smooth internal surface and external surface, designed to prevent trapping of debris. The trocar sleeve excludes narrow features (e.g., narrow passages, small cavities) that are likely to trap debris. The trocar sleeve is designed for flow of washing fluids to flow through for removal of debris. One or more dimensions of the trocar sleeve may be different in comparison to trocar sleeves designed for single-use.

At least some implementations of the trocar sleeve described herein address the technical problem of reducing biological waste from medical procedure. Standard trocars are designed to be single-use and disposed of. The multiple trocars used per surgical procedure, multiplied by the number of procedures performed daily, generates a large amount of biological waste. Such waste, which is a biological hazard cannot be recycled, making it difficult to dispose of. At least some implementations of the trocar sleeve described herein improve the technical field of medical devices, by reducing waste of the medical devices.

At least some implementations described herein address the aforementioned technical problem, and/or improves upon the aforementioned technical field, by providing trocar sleeves that can be used multiple times, which greatly reduces biological waste generated from single-use trocar sleeves. The multi-use of the trocar sleeves is enabled by the material selection and/or surface design in comparison to single-use trocar sleeves. The trocar sleeve designed for multi-use is made of a biocompatible material designed to withstand multiple sterilization cycles, which is different than a biocompatible material that is designed for single use, which does not need to undergo any sterilization cycles, or undergoes a single sterilization cycle for an initial sterilization after manufacturing but without being used, which may be different than sterilization after use. The internal and/or external surface of the trocar sleeve may be designed to enable sterilization, by preventing debris from being stuck and/or enabling debris to be washed out by washing fluid. Remaining debris prevent sterilization. The internal surface and/or external surface are smooth. The internal surface and/or external surface exclude narrow features that trap debris, which enable washing fluids to flow through for removal of debris. In contrast, single-use trocar sleeves have such narrow features that trap debris, even after washing. The debris trapped in the narrow features prevent sterilization. One or more dimensions of the trocar sleeve may be larger in comparison to trocar sleeves designed for single-use. The larger dimension(s) enable washing fluid to wash out debris, which enables safe and efficient sterilization. In contrast, single-use trocar sleeves with smaller dimensions may trap debris, which cannot be fully removed by the washing fluid. The trapped debris prevent sterilization.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

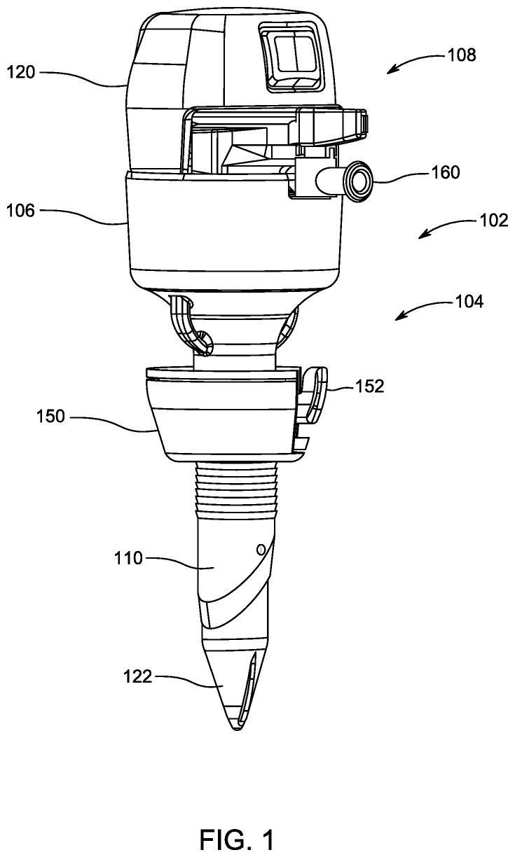

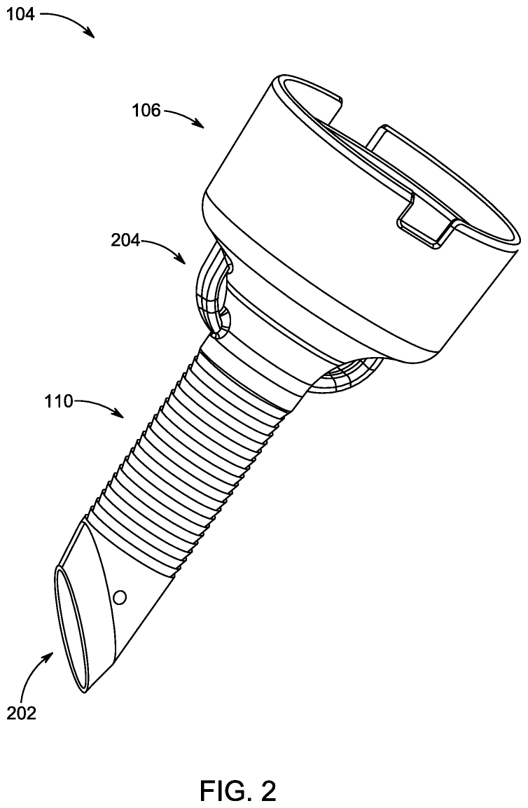

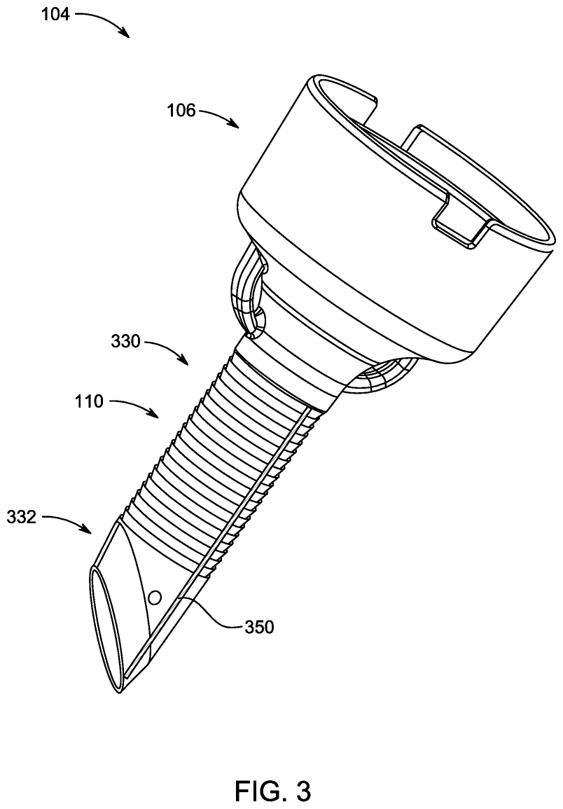

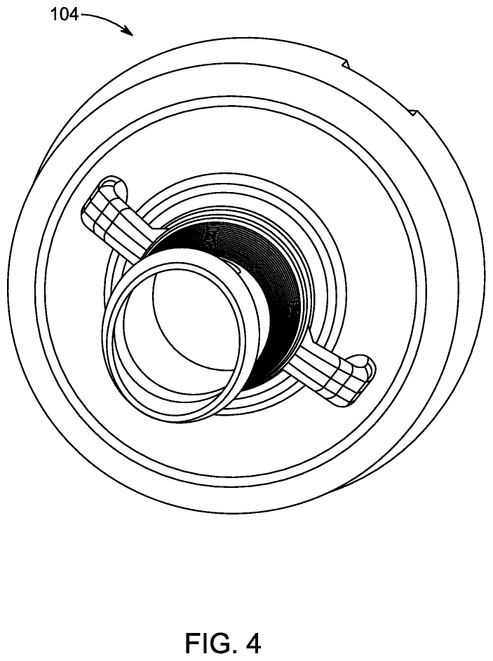

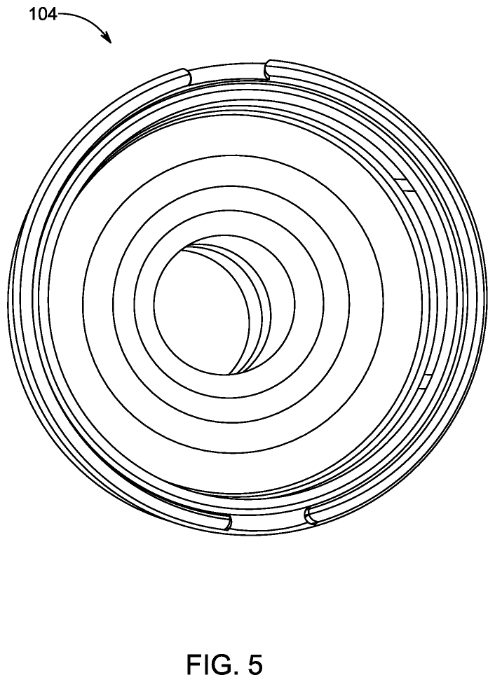

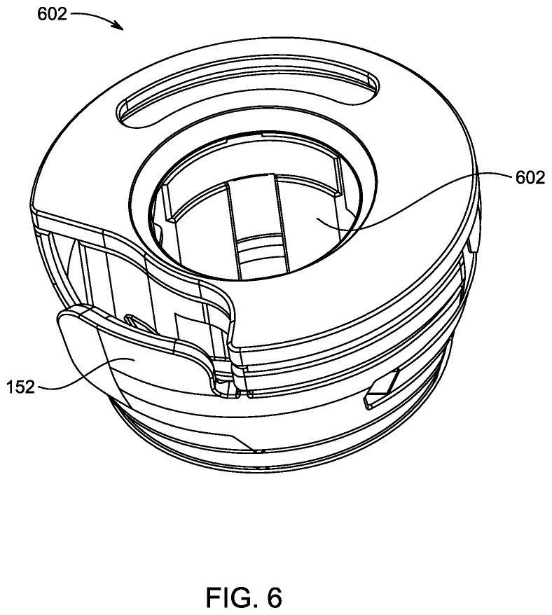

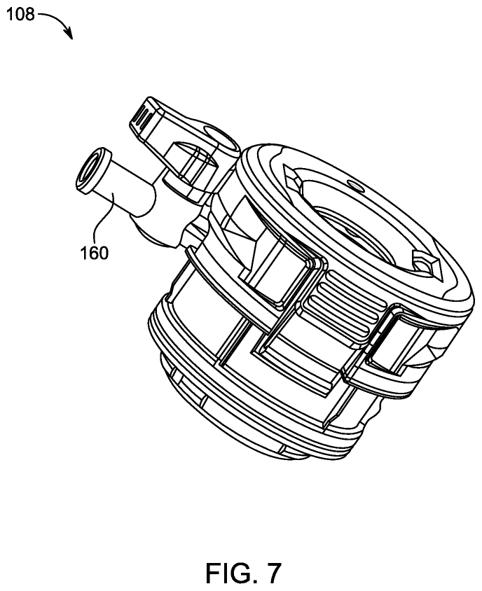

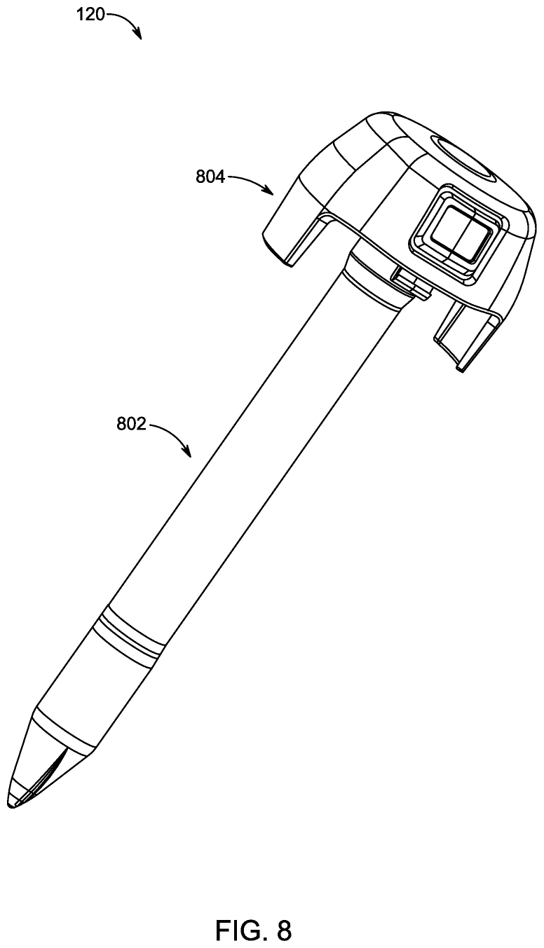

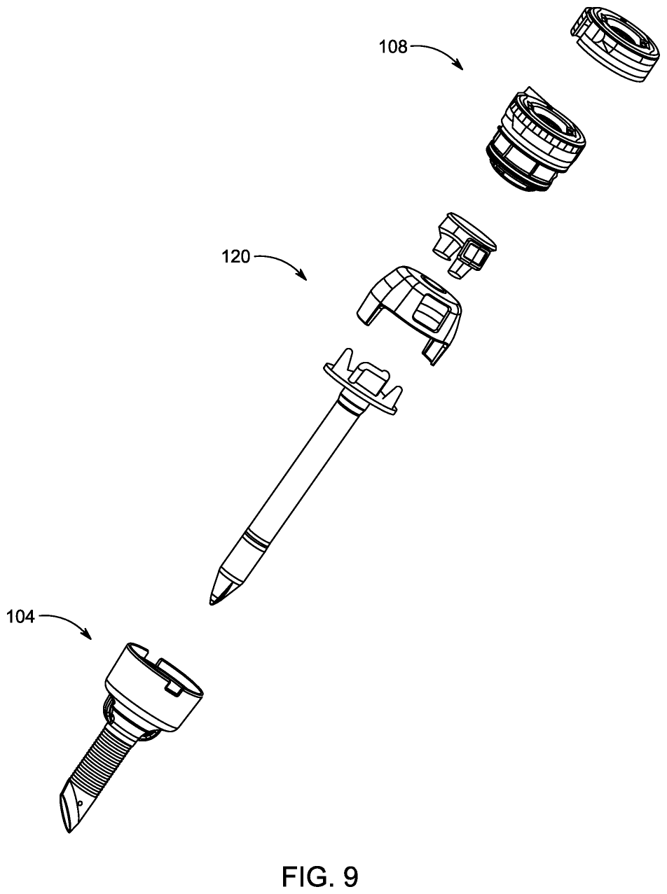

Reference is now made to FIG. 1, which is a schematic of a trocar assembly 102 that includes a trocar sleeve 104 designed for multiple uses, in accordance with some embodiments of the present invention. Reference is also made to FIG. 2, which is a schematic of an exemplary trocar sleeve 104 designed for multiple uses, in accordance with some embodiments of the present invention. Reference is also made to FIG. 3, which is a schematic of an exemplary trocar sleeve 104 designed for multiple uses and designed to engage with depth limiter 150, in accordance with some embodiments of the present invention. Reference is also made to FIG. 4, which is a schematic of a bottom view (i.e., from distal to proximal) of trocar sleeve 104 designed for multiple uses, in accordance with some embodiments of the present invention. Reference is also made to FIG. 5, which is a schematic of a top view (i.e., from proximal to distal) of trocar sleeve 104 designed for multiple uses, in accordance with some embodiments of the present invention. Reference is also made to FIG. 6, which is a schematic of an exemplary depth limiter 150, in accordance with some embodiments of the present invention. Reference is also made to FIG. 7, which is a schematic of an exemplary sealing element 108 that engages with trocar sleeve 104, in accordance with some embodiments of the present invention. Reference is also made to FIG. 8, which is a schematic of an exemplary obturator 120 that engages with trocar sleeve 104, in accordance with some embodiments of the present invention. Reference is also made to FIG. 9, which is a schematic of an exploded view of trocar assembly 102, in accordance with some embodiments of the present invention.

Referring now back to FIG. 1, trocar assembly 102 includes trocar sleeve 104. Trocar sleeve 104 includes a trocar housing 106 and trocar cannula 110. Trocar sleeve 104 includes a lumen designed to engage with an obturator 120.

Trocar assembly 102 may include a depth limiter component 150 placed along a shaft of a trocar cannula 110. Depth limiter 150 includes a lumen sized and shaped to accommodate trocar cannula 110, and a locking mechanism 152 that sets trocar cannula 110 in a fixed position within the lumen, and/or that sets depth limiter 150 at a fixed position along trocar cannula 110. The internal diameter of the lumen of depth limiter 150 is slightly larger than the outer diameter of trocar cannula 110, to enable sliding depth limiter 150 along a length of trocar cannula 110. An exemplary depth limiter component 150 is described for example, with reference to U.S. Patent Provisional Patent Application entitled “TROCAR ASSEMBLY”, Attorney Docket No. 94013, the contents of which are incorporated herein by reference in their entirety.

Obturator 120 may be bladed or non-bladed. Obturator 120 may include a piercing tip 122 which is used to pierce the skin of the subject until a cavity is entered, for example, the abdominal cavity. After penetration into the abdominal cavity is complete, obturator portion 120 may be removed from trocar cannula 110. After the obturator portion is removed, any number of surgical instruments such as, for example, a tissue fastening instrument can be inserted through the cannula 110 of the trocar assembly 102 to perform a surgical procedure.

Trocar assembly 102 may include a sealing element 108 designed to engage trocar housing 106. Sealing element 108 may be located within obturator 120, and/or within housing 106, and/or other designs may be implemented. Sealing element 108 may be designed to prevent or reduce the escape of fluid and/or gas during endoscopic procedures. During an endoscopic surgical procedure, the internal gas pressure in the body cavity (e.g., abdomen) is to be maintained above ambient pressure in order to successfully complete the procedure. Sealing element 108 is designed to maintain gas pressure in the body cavity (e.g., abdominal cavity), despite numerous insertions and withdrawals of surgical instruments through the trocar cannula.

One or more components of trocar assembly 102 may be designed to be reusable in multiple surgical procedures, by having surfaces that are designed to enable washing fluids to wash debris for avoiding retaining of debris between washing cycles, for example smooth surfaces, and/or lack of narrow passages and/or narrow cavities where the debris may be retained. The components of trocar assembly 102 are made of a material designed to be reusable are designed to withstand two or more washing and/or sterilization cycles, for example, at least about 100, or 250, or 500, or 1000, or other values. For example, PEEK, and PPSU. Components of trocar assembly 102 designed to be reusable include one or more of: cannula 110, trocar housing 106, trocar sleeve 104, depth limiter 150, and/or obturator 120. It is noted that sealing element 108 may be disposable, due to the design of sealing element 108 that is prone to retaining debris, and therefore cannot be reliably washed and sterilized multiple times.

Trocar assembly 102 may include a valve 160 (e.g., stop-cock design). Valve may be integrated within sealing element 108, for example, as shown with reference to FIG. 7. Valve 160 may be integrated within sealing element 108 for being positioned at the proximal end of the trocar housing 106. When trocar assembly 102 is assembled, valve 160 may be positioned in communication with trocar housing 106 for selectively allowing and/or preventing the passage of an insufflation fluid, for example carbon dioxide, into an annular space formed between an internal diameter of trocar housing 106 and obturator 120 and/or another instrument placed within lumen of trocar housing 106.

Referring now back to FIG. 2, trocar sleeve 104 includes cannula 110 attached to housing 106. Cannula 110 has a distal end and a proximal end. Housing 106 has a distal end and a proximal end. The proximal end of housing 106 is attached to the distal end of cannula 110, for example, as a single piece (e.g., manufactured by injection molding), by screwing one component into the other component, by gluing, crimping, and the like.

A lumen 202 within trocar sleeve 104 extends from the distal end of cannula 110 to the proximal end of housing 106. Lumen 202 is designed to accommodate obturator 120. An internal diameter of lumen 202 is selected to provide a clearance of about 0.2 millimeters (mm), or about 0.3 mm, or about 0.5 mm, or about 0.2-0.5 mm, or other values, from an outer diameter of obturator 120 located within 202 lumen.

Trocar sleeve 104 (i.e., cannula 110 and housing 106) is made of a biocompatible material designed to withstand multiple sterilization cycles, for example, PEEK, and PPSU. The biocompatible material is designed to withstand two or more sterilization cycles, for example, at least about 50, or 100, or 250, or 500, or 1000, or any other number of sterilization cycles. The biocompatible material is designed to withstand sterilization cycles performed, for example, by an autoclave device. Trocar sleeve 104 has a smooth internal surface and external surface, including an internal surface of lumen 202, that excludes narrow features such as small cavities and/or narrow indentations that trap debris. Trocar sleeve 104 is designed for flow of washing fluids to flow through for removal of debris, for example, washing fluids may flow into one end of lumen 202 and out the other end of lumen 202, and/or across the outer surface of sleeve 104. One or more dimensions of the trocar sleeve may be larger in comparison to trocar sleeves designed for single-use. For example, the inner diameter of lumen 202 may be larger in comparison to single-use trocar sleeves, to enable washing fluids to flow through lumen 202 for preventing debris from being retained inside. Preventing debris from becoming stuck in trocar sleeve enables sterilizing trocar sleeve 104 for multiple uses. Remaining debris prevent sterilization.

Exemplary dimensions of trocar sleeve 104 include:

* An internal diameter of lumen 202 of trocar sleeve 104 is set according to the external diameter of the obturator and/or instrument that is expected to be passed through lumen 202. For the obturator and/or instrument with an external diameter of 5 mm, the internal diameter of lumen 202 is 7.1-7.3 mm. For the obturator and/or instrument with an external diameter of 12 mm, the internal diameter of lumen 202 is 14.1-14.3 mm.

Lumen 202 may have a constant diameter within trocar sleeve 104, including cannula 110 and/or housing 106. Alternatively, lumen 202 has a constant diameter within cannula 110. The diameter of lumen 202 within cannula may match an internal diameter of sealing element 108 when placed within housing 106, for maintaining an overall constant diameter of an internal diameter when trocar assembly 102 is assembled.

The internal diameter of lumen 202 may be designed to increase the cross sectional area of the annular space formed between the internal diameter of lumen 202 of trocar sleeve 104 and an external diameter of obturator 120 and/or another instrument passed through lumen 202. The cross sectional area of the annular space may be increased in comparison to other known trocar cannulas, for increasing the insufflation rate (e.g., gas flow, optionally carbon dioxide gas) into the body cavity (e.g., abdomen).

* An outer diameter of cannula 110 is set according to the external diameter of the obturator and/or instrument that is expected to be passed through lumen 202. For the obturator and/or instrument with an external diameter of 5 mm, the outer diameter of cannula 110 is 9.45-10.6 mm. For the obturator and/or instrument with an external diameter of 12 mm, the outer diameter of cannula 110 is 16.75-17.7 mm.

* An outer diameter of housing 106 ranges from 44.8-47.0 mm.

* A working length of the trocar sleeve 104 may be in the range of about 60 mm-150 mm, for example, 60 mm, 75 mm, 100 mm, and 150 mm. Other exemplary ranges include 50 mm-160 mm, and 45 mm-155 mm. In some embodiments, the working length of trocar sleeve 104 is shorter than other known trocar lengths. The short working length is designed for pediatric use, for example, 45 mm-70 mm, or 50-60 mm, or about 45 mm, 50 mm, 55 mm, 60 mm, 65 mm. The short working length reduces risk of injury in pediatric subjects, from the distal end of the trocar contacting internal tissues. It is noted that standard (non-pediatric) lengths may be used with the depth limiter described herein for preventing injury. Pediatric lengths may be used with the depth limiter as a multi-layer safety net for preventing injury.

Trocar sleeve 104 may include one or more protruding features 204 designed for securing thread that is stitched to skin of a subject when trocar sleeve 104 is in use and positioned through the skin. Protruding features 204 are designed to enable washing fluid to wash through, and/or include smooth surfaces, and/or exclude narrow indentations, for preventing retention of debris to allow sterilization. Protruding features 204 may include, for example, rings and handles. Optionally, protruding features 204 include one or more handles attached to a proximal region of cannula 110 and a distal region of housing 106.

Referring now back to FIG. 3, optionally, cannula 110 includes one or more raised features 330 on its outer surface that are designed to be engaged by the locking mechanism 152 of depth limiter 150, for example, spaced apart elevated rings where the lock includes a C-shaped element that falls between the elevated rings, or ratchets where the locking mechanism 152 includes a pawl designed to allow easy removal of the cannula but prevent pushing the cannula deeper unless a leaver is pressed to release the pawl.

Optionally, a distal end 332 of cannula excludes raise features 330, to prevent the user from fixing depth limiter 150 at a position that is too low along cannula 110, where depth limiter 150 may not be able to properly support and/or engage cannula 110.

In another exemplary implementation, raised features 330 may be set at varying diameters, for example, sequentially increasing, or sequentially decreasing. For example, to enable preselection of a maximum depth, which may prevent distal pushing of the cannula and/or may enable removal. Alternatively or additionally, an external diameter of cannula 110 varies across a length of cannula 110. The external diameter may decrease from the proximal end of the cannula to the distal end of the cannula. For example, locking mechanism 152 is set on a first diameter of cannula 110. Proximally along cannula 110, the diameter increases, which prevents pushing cannula 110 distally into the body of the subject. Locking mechanism 152 may be preset to the first diameter before cannula 110 is inserted into the lumen of depth limiter 150, which enables the user to blindly insert cannula 110 at the predefined depth by inserting cannula 110 into the preset locking mechanism 152 of depth limiter 150.

Optionally, raised features 330 are spaced apart along a long axis of cannula 110 for defining a resolution for fixing the location of cannula 110 relative to the depth limiter 150.

Alternatively, the external surface of cannula 110 is smooth, excluding raised features 330, for example, in implementations in which depth limiter 150 is connected to cannula 110. The smooth exterior surface of cannula 110 reduces damage to tissue during insertion, and/or reduces required pressure to insert cannula 110 into the body. In prior approaches, surface features may be designed to anchor cannula 110 within the tissue. Using depth limiter 150 prevents cannula 110 from further penetrating into the body, which may make surface features of prior approaches irrelevant.

Optionally, an external surface of cannula 110 includes one or more guide elements 350 set for engaging corresponding guide element(s) of depth limiter 150. Cannula 110 is insertable into a lumen of depth limiter 150 by engaging the guide element(s) 350 of cannula 110 with the corresponding guide element(s) of depth limiter 150. A mismatch of the guide elements 350 of cannula 110 and depth limiter 150 prevents trocar cannula 110 from being inserted into the lumen of the depth limiter 150. Once guide element(s) 350 of cannula 110 engage the guide element(s) of depth limiter 150, rotation of cannula 110 within depth limiter 150 is prevented.

Guide element(s) 350 of cannula 110 and corresponding guide element(s) of depth limiter 150 may be implemented, for example, as a rail and corresponding trench sized to match the rail, and/or a non-circular shape of an external surface of cannula 110 and corresponding non-circular shape of an internal surface of the lumen of depth limiter 150 such as a square, oval, and four leaf clover.

Referring now back to FIG. 4, the bottom view (i.e., from distal to proximal) of trocar sleeve 104 designed for multiple uses, is depicted.

Referring now back to FIG. 5, the top view (i.e., from proximal to distal) of trocar sleeve 104 designed for multiple uses, is depicted.

Referring now back to FIG. 6, depth limiter 150 includes a locking mechanism 152 that secures cannula 110 within a lumen 602. Locking mechanism 152 prevents cannula 110 from moving proximally and/or distally within lumen 602 of depth limiter 150. In use, when cannula 110 is located in the body of the patient, depth limiter 150 sets a maximum depth of cannula 110, for example, preventing cannula 110 from entering deeper into the body and injuring tissues.

Referring now back to FIG. 7, exemplary sealing element 108 may be engaged with trocar sleeve 104. Trocar sleeve 104 is designed for engaging with sealing element 108. Sealing element 108 may be single-use, due to the design that includes narrow features that are prone to trapping debris that cannot be washed out, which prevents sterilization.

Referring now back to FIG. 8, exemplary obturator 120 may be engaged with trocar sleeve 104. Trocar sleeve 104 is designed for engaging with obturator 120. Obturator 120 may be designed for multiple uses, made of biocompatible material designed to withstand multiple sterilization cycles, and/or including smoother surfaces and/or excluding narrow features that are prone to trapping debris that enable sterilization. Optionally, an external diameter of a shaft 802 of obturator 120 may be, for example, (e.g., about) 46 mm when designed to fit into a trocar cannula and/or sleeve with an internal lumen diameter of (e.g., about) 50 mm. A head 804 of obturator 120 may be larger than standard sizes. Head 804 may be more dense and/or weigh more than standard obturators. The larger size, increased density, and/or higher weight may provide a larger mass that provides a larger moment for the user (e.g., surgeon).

Referring now back to FIG. 9, the exploded view depicts an exemplary approach for assembling trocar assembly 102, from trocar sleeve 104, obturator 120, and sealing element 108. It is noted that depth limiter 150 is not shown.

It is expected that during the life of a patent maturing from this application many relevant trocars will be developed and the scope of the term trocar is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A trocar sleeve comprising:
a cannula having a distal end and a proximal end;
a housing having a distal end and a proximal end, the proximal end of the housing attached to the distal end of the cannula; and
a lumen extending from the distal end of the cannula to the proximal end of the housing,
wherein the cannula and housing are made of a biocompatible material designed to withstand a plurality of sterilization cycles.

2. The trocar sleeve of claim 1, wherein the biocompatible materials is selected from a group comprising: polyetheretherketone (PEEK), and polyphenylsulfone (PPSU).

3. The trocar sleeve of claim 1, wherein the biocompatible materials is designed to withstand at least two sterilization cycles.

4. The trocar sleeve of claim 1, wherein the trocar sleeve has a smooth internal surface and external surface that excludes narrow features that trap debris and designed for flow of washing fluids to flow through for removal of debris for sterilization.

5. The trocar sleeve of claim 1, wherein a range of an internal diameter of the lumen is 7.1-7.3 mm for an obturator or instrument with an external diameter of 5 mm, and 14.1-14.3 mm for an obturator or instrument with an external diameter of 12 mm.

6. The trocar sleeve of claim 1, wherein an internal diameter of the lumen is selected to provide a clearance of about 0.3 mm from an outer diameter of an obturator placed within the lumen.

7. The trocar sleeve of claim 1, wherein a range of an outer diameter of the cannula is 9.45-10.6 mm for an obturator or instrument with an external diameter of 5 mm, and 16.75-17.7 mm for an obturator or instrument with an external diameter of 12 mm.

8. The trocar sleeve of claim 1, wherein an outer diameter of the housing ranges from 44.8-47.0 mm.

9. The trocar sleeve of claim 1, wherein a working length of the trocar sleeve ranges from about 45 mm to 65 mm for pediatric use.

10. The trocar sleeve of claim 1, wherein a working length of the trocar sleeve ranges from 60-150 mm.

11. The trocar sleeve of claim 1, further comprising at least one protruding feature designed for securing thread that is stitched to skin of a subject when the trocar sleeve is in use and positioned through the skin.

12. The trocar sleeve of claim 1, wherein the cannula includes a plurality of raised features designed to engage a locking mechanism of a depth limiter that fixes location of the cannula relative to the depth limiter.

13. The trocar sleeve of claim 12, wherein a distal end portion of the cannula excludes the raised features.

14. The trocar sleeve of claim 12, wherein the plurality of raised features are spaced apart along a long axis of the cannula defining a resolution for fixing the location of the cannula relative to the depth limiter.

15. The trocar sleeve of claim 1, wherein an external diameter of the cannula varies across a length of the cannula, the external diameter decreases from the proximal end of the cannula to the distal end of the cannula.

16. The trocar sleeve of claim 1, wherein an external surface of the cannula includes a first guide element set for engaging a corresponding second guide element of a depth limiter, wherein the cannula is insertable into a lumen of the depth limiter by engaging the first guide element with the second guide element, and the trocar cannula is prevented from being inserted into the lumen of the depth limiter when the first guide element does not engage the second guide element.

17. The trocar sleeve of claim 16, wherein the first guide element engaging the second guide element prevent rotation of the cannula within the depth limiter.
